(19) United States
(12) Patent Application Publication
(10) Pub. No.: US 2024/0390105 A1
(43) Pub. Date: Nov. 28, 2024

(54) APPARATUS AND SYSTEM FOR ALIGNING A MEDICAL OBJECT WITH RESPECT TO AN EXAMINATION OBJECT, AND METHOD FOR EMITTING A LIGHT DISTRIBUTION BY A LIGHT GUIDING FACILITY

(71) Applicant: Siemens Healthineers AG, Forchheim (DE)

(72) Inventor: Alois Regensburger, Poxdorf (DE)

(21) Appl. No.: 18/674,858

(22) Filed: May 25, 2024

(30) Foreign Application Priority Data
May 25, 2023 (DE) 10 2023 204 909.0

(51) Int. Cl. A61B 90/00

(52) U.S. Cl. CPC A61B 90/39; A61B 90/37; A61B 2090/363; A61B 2090/373; A61B 2090/376; A61B 2090/3945; A61B 2090/3983

(57) ABSTRACT

An apparatus for aligning a medical object with respect to an examination object includes an alignment element that may be fastened on or integrated into the medical object, having a number of at least point-shaped markers. The apparatus includes a processing unit configured to receive medical image data and identify positioning information relating to a positioning of the medical object. The apparatus includes a representation unit configured to show a graphical representation of the medical image data and an item of alignment information. The alignment information relating to the markers has a corresponding virtual continuation of the medical object in each case, which corresponds to virtual arrangements of the medical object. The apparatus includes a light guiding facility configured to emit a predefined light distribution such that one of the markers is illuminated by the light distribution when the medical object is aligned according to the corresponding virtual continuation.

This application claims the benefit of German Patent Application No. DE 10 2023 204 909.0, filed on May 25, 2023, which is hereby incorporated by reference in its entirety.

### BACKGROUND

The present embodiments relate to an apparatus for aligning a medical object with respect to an examination object, a system, a method for emitting a light distribution, and a computer program product.

Within the scope of minimally invasive interventions (e.g., bone interventions, such as vertebroplasty and/or kyphoplasty), a bone trocar, a K-wire, a stiff needle, and/or a screw-like tool as an intervention instrument is frequently introduced into a bone structure of an examination object (e.g., by hammering and/or drilling). The intervention instrument may frequently be guided via an entry point on the examination object to a target object. For this purpose, a monitoring of a positioning (e.g., an alignment and/or position) of the intervention instrument with respect to the examination object is often essential. Fluoroscopy is often used here for intraprocedural navigation and/or guidance of the intervention instrument (e.g., by a C-arm x-ray device). In most cases, planning imaging and/or 3 D imaging is not carried out preinterventionally.

If necessary, a positioning of the intervention instrument and/or an angulation of the C-arm x-ray device may be adjusted using the fluoroscopy imaging, for example. Alternatively or in addition, the intervention instrument (e.g., a longitudinal extension direction of the intervention instrument) may be detected in the fluoroscopy images and shown by a representation unit as a graphical representation by overlaying a straight line. By iterative repositioning of the intervention instrument using fluoroscopy imaging, the overlaid straight light may be aligned with the target object. The known methods disadvantageously result in an increased radiation exposure of the examination object. Further, the methods are often time-consuming.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an alignment of a medical object with respect to an examination object that has a low x-ray dose and is time-efficient, based on a two-dimensional (2 D) imaging is provided.

Independent of the grammatical term usage, individuals with male, female or other gender identities are included within the term.

The present embodiments relate, in a first aspect, to an apparatus for aligning a medical object with respect to an examination object. The apparatus includes an alignment element, a light guiding facility, a processing unit, and a representation unit. The alignment element has a number of at least point-shaped markers and may be fastened on the medical object or integrated into the medical object in a defined positional relationship. Further, the processing unit is configured to receive medical image data, having a mapping of the examination object. Further, the processing unit is configured to identify an item of positioning information relating to a positioning of the medical object. The representation unit is configured to show a graphical representation of the medical image data and an item of alignment information as a function of the positioning information. The alignment information has a corresponding virtual continuation of the medical object with respect to the markers. The virtual continuations may correspond to virtual arrangements of the medical object. Further, the light guiding facility is configured to emit a predefined light distribution. In an operating state of the apparatus, the medical object is arranged on or in the examination object. Further, in the operating state, the representation unit displays the graphical representation of the medical image data and the alignment information. Further, in the operating state, the light guiding facility sends the light distribution such that one of the markers is illuminated by the light distribution when the medical object is aligned according to the corresponding virtual continuation.

The examination object may be, for example, a human and/or animal patient and/or a human and/or animal patient and/or an examination phantom.

The medical object may be a surgical instrument (e.g., a needle, such as a puncture needle), and/or a trephine, and/or a diagnostic instrument (e.g., an endoscope, such as a laparoscope), and/or a catheter, and/or a trocar. In one embodiment, the medical object may be configured at least in part (e.g., completely) to be rigid and elongated (e.g., in a rod and/or needle-shaped manner).

In one embodiment, the medical object (e.g., a distal section of the medical object, in the operating state of the apparatus) may be arranged on or in the examination object. For example, the medical object in the operating state of the apparatus may be arranged at least in part on or in the examination object.

The alignment element may be fastened (e.g., arranged) in a defined positional relationship (e.g., in a defined spatial relative position and/or relative alignment and/or relative pose) on the medical object (e.g., a proximal section of the medical object). For example, the alignment element may be detachably fastened on the medical object in the defined positional relationship. For this purpose, the alignment element and/or the medical object may have a fastening element (e.g., a clamping apparatus and/or a plug connection and/or a magnetic holder). Alternatively, the alignment element may be integrated into the medical object (e.g., at a proximal section of the medical object, such as on a surface of the medical object).

The alignment element may have a number of at least point-shaped markers in a defined arrangement. The number of at least point-shaped markers may be configured as geometric objects (e.g., line and/or circle and/or cross). Further, the number of markers may be configured as a contrast object (e.g., imprint and/or embossment and/or cutout on a substrate of the alignment element, such as a surface of the alignment element).

The processing unit may be configured to receive the medical image data. The receiving of the image data may include, for example, a capture and/or reading-out of a computer-readable data store and/or a receiving from a data storage unit (e.g., a database). For this purpose, the processing unit may have a corresponding interface. In one embodiment, the image data may be provided by a medical imaging device for recording the image data.

In one embodiment, the image data has a two-dimensional or three-dimensional spatially resolved mapping of the examination object. Further, the image data may map the examination object in a time-resolved manner. The image data may have a number of image points (e.g., pixels or voxels) with image values (e.g., intensity values and/or attenuation values) that map (e.g., represent) the examination object. The image data may have, for example, x-ray projection mappings (e.g., fluoroscopy images) of the examination object.

Further, the processing unit may be configured to provide the positioning information having an item of information relating to the identified positioning of the medical object on the light guiding facility (e.g., using the interface). The provision of the positioning information may include a storage on a computer-readable storage medium and/or a transmission to the light guiding facility (e.g., an interface of the light guiding facility).

The representation unit may include a screen and/or monitor and/or projector and/or smart glasses, which are configured to display the graphical representation of the medical image data and the alignment information. The alignment information may have a virtual continuation of the medical object that corresponds to the markers in each case.

The virtual continuations may correspond to a virtual arrangement (e.g., alignment and/or pose) of the medical object (e.g., about a distal reference point of the medical object) in each case. In one embodiment, the virtual arrangements of the medical object may describe different (e.g., non-symmetrical) alignments of the medical object (e.g., about the distal reference point, such as within a plane). The graphical representation may represent the virtual continuations as graphical elements (e.g., a line and/or arrow). For example, the graphical representation may have the virtual continuations as an at least partial overlay of the image data. In one embodiment, the processing unit may be configured to adjust the alignment information (e.g., a representation parameter and/or a number of virtual continuations) as a function of the positioning information. By way of example, the processing unit may adjust a distance and/or angle of the virtual continuations in the graphical representation as a function of the positioning information such that one of the markers is illuminated by the light distribution when the medical object, according to the corresponding virtual continuation, is aligned, for example, in a rotationally independent manner with respect to the longitudinal extension direction and/or object axis of the medical object. The object axis may include an axis of symmetry and/or longitudinal axis and/or progression axis and/or advance axis of the medical object.

In the operating state of the apparatus, the representation unit may display the graphical representation of the medical image data and the alignment information.

The light guiding facility may include a light source (e.g., a laser light source) that is configured to emit the predefined light distribution. For this purpose, the light guiding facility may include an optical diaphragm. In one embodiment, the light guiding facility may be configured to emit the predefined light distribution as a function of the positioning information. For example, the light guiding facility may be configured to adjust a projection direction and/or projection geometry of the predefined light distribution as a function of the positioning information.

In one embodiment, the light guiding facility may emit the light distribution in the operating state of the apparatus such that one of the markers (e.g., precisely one of the markers) is illuminated through the light distribution, when the medical object is aligned according to the virtual continuation that corresponds to the illuminated marker.

The light guiding facility may emit the light distribution, for example, in the operating state of the apparatus such that a light pattern (e.g., a line and/or a cross and/or a point) is projected onto the marker.

The alignment information may further have a virtual representation of the light pattern (e.g., a line and/or a cross and/or a point).

Using the apparatus of the present embodiments, an alignment of the medical object that has a low x-ray dose and is time-efficient with respect to the examination object may be enabled based on the image data (e.g., without a 3 D imaging and/or a navigation system and/or a path planning). In this way, it is possible to dispense with a registration of the image data with a planning mapping of the examination object. Further, repositionings and/or corrections of a positioning of the medical object may be minimized. In one embodiment, an intuitive selection of the alignment of the medical object by a medical operating personnel (e.g., a physician) may be enabled using the graphical representation of the medical image data and the alignment information (e.g., the virtual continuations). Further, at the same time, the light guiding facility may be configured to emit a further light distribution in order to assist with the medical operating personnel when positioning the examination object.

In a further embodiment of the apparatus, the markers may have an optically distinguishable property. Further, the virtual continuations in the graphical representation of the alignment information may have an optically distinguishable property. The optically distinguishable property of the virtual continuations may correspond in pairs to the optically distinguishable property of one of the markers in each case.

The number of markers may have a color coding and/or black-white coding and/or a surface condition (e.g., a reflectivity and/or fluorescence and/or contour) as the optically distinguishable property, for example. In one embodiment, the number of markers may be clearly identifiable using the optically distinguishable property.

Further, the virtual continuations in the graphical representation of the alignment information (e.g., the graphical representation of the virtual continuations) may have an optically distinguishable property (e.g., a color coding and/or black-white coding and/or a line style and/or a line width and/or a line shape and/or an inscription).

In one embodiment, the optically distinguishable property of the virtual continuations may correspond in pairs (e.g., by matching colors) in each case to the optical distinguishable property of the markers. For example, a mapping that maps the optically distinguishable property of the virtual continuations one-to-one onto the optically distinguishable property of the markers may exist.

The embodiment may enable an improved capture of the markers that correspond to the virtual continuations in each case.

In a further embodiment of the apparatus, the markers may be configured as 2 D lines within a plane in each case. The planes intersect along a shared straight line that forms a movement axis of the medical object with respect to the examination object and not an object axis of the medical object.

In one embodiment, the number of markers may be configured in each case as, for example, straight or curved 2 D lines that run within a plane in each case. For example, each of the number of markers may be configured as a 2 D line within a plane in each case, where the planes are different. The number of markers may be configured as 2 D lines (e.g., graphical 2 D lines) and/or cutouts and/or elevations and/or contours. The planes, within which the 2 D lines run, may intersect (e.g., cross) along the shared straight line. For example, the shared straight line may form a line of intersection of the number of planes.

The shared straight line may form a movement axis of the medical object with respect to the examination object. For example, the medical object may be able to be rotated and/or tilted about the shared straight line. The shared straight line may run through the reference point (e.g., a distal reference point of the medical object) and/or a distal section of the medical object, for example. With an elongated embodiment of the medical object, the shared straight line may run essentially at a right angle to a longitudinal extension direction of the medical object. In one embodiment, the shared straight line does not form an object axis (e.g., an axis of symmetry and/or longitudinal axis and/or progression axis and/or advance axis) of the medical object.

In one embodiment, the image data may map the medical object in parallel with the shared straight line.

The embodiment may enable an intuitive alignment of the medical object with respect to the examination object by tilting and/or rotating the medical object about the shared straight line.

In a further embodiment of the apparatus, the number of markers may be arranged essentially on a shared (e.g., flat) side of the alignment element.

In one embodiment, the alignment element may have a flat (e.g., planar and/or level) or curved (e.g., convex or concave) side. The number of markers may be arranged on a surface of the shared (e.g., flat or curved) side of the alignment element or integrated at least in part (e.g., completely) into the surface of the shared side of the alignment element.

The embodiment may enable a particularly simple projection and capture of the predefined light pattern on the surface of the shared side of the alignment element.

In a further embodiment of the apparatus, the light guiding facility may emit the light distribution in the operating state so that a shared line is projected. The light distribution illuminates one of the markers and a reference point of the medical object when the medical object is aligned according to the corresponding, virtual continuation.

In one embodiment, the light guiding facility, in the operating state of the apparatus, may project a straight line (e.g., a route). With an alignment of the medical object according to one of the virtual continuations, the projected straight line may illuminate the corresponding marker and the reference point. The reference point may be arranged on the medical object (e.g., on a distal section of the medical object). Alternatively or in addition, the reference point may be arranged on a further marker, so that the marker corresponding to the virtual continuation and a further marker corresponding in particular to none of the virtual continuations are illuminated by the straight light when the medical object is aligned according to the virtual continuation.

The embodiment may enable a precise alignment of the medical object based on the projected straight line.

In a further embodiment of the apparatus, the medical object may be configured to be essentially rigid and elongated. The reference point may be arranged along a longitudinal extension direction of the medical object.

In one embodiment, the medical object may be configured to be essentially rigid (e.g., not flexible and/or not or only marginally deformable). Further, the medical object may be configured to be elongated (e.g., rod-shaped and/or needle-shaped). In an embodiment of the medical object as a trocar (e.g., bone trocar), the medical object may have a length of 10 cm to 30 cm or over 20 cm, for example. For example, the medical object may have a straight longitudinal extension direction. The reference point may be arranged along the longitudinal extension direction (e.g., on the distal section, such as a tip and/or a distal end section) of the medical object. With the arrangement of the medical object on or in the examination object in the operating state of the apparatus, the reference point may be a contact point of the medical object with the examination object (e.g., a hard structure, such as a bone surface and/or a bone structure). Further, the reference point in the operating state may be arranged inside or outside of the examination object.

The image data may map the medical object essentially at a right angle to the longitudinal extension direction of the medical object.

The embodiment may enable a precise alignment (e.g., fine adjustment of the alignment) of the medical object about the reference point. The medical object may be aligned so that the virtual continuation and the medical object are aligned in the direction of a target object of the examination object.

In a further embodiment of the apparatus, the light guiding facility may emit the predefined light distribution in the operating state as a function of the positioning information.

The light guiding facility may be configured to adjust the emission of the predefined light distribution (e.g., a projection geometry) as a function of the positioning information. By way of example, the light guiding facility may adjust a projection direction and/or a projection angle and/or a projection geometry and/or a focusing of the predefined light distribution as a function of the positioning information.

By the emission of the predefined light distribution that is adjusted as a function of the positioning information, it is possible to provide that one of the markers is illuminated by the light distribution when the medical object is aligned according to the corresponding virtual continuation (e.g., also after a repositioning of the medical object).

The present embodiments relate, in a second aspect, to a system including an apparatus of the present embodiments and a medical imaging device. The imaging device is configured to record medical image data.

The medical imaging device for recording the image data may include a medical x-ray device (e.g., a medical C-arm x-ray device and/or a cone-beam computed tomography system (cone-beam CT, CBCT) and/or a computed tomography system (CT system) and/or a magnetic resonance tomography system (MRT system) and/or a positron emission tomography system (PET system) and/or an ultrasound device). In one embodiment, the imaging device may be configured to record and provide the image data.

The advantages of the system of the present embodiments correspond essentially to the advantages of the apparatus of the present embodiments. Features, advantages, or alternative embodiments mentioned here may likewise also be transferred to the other claimed subject matters and vice versa.

In a further embodiment of the system, the imaging device may have an x-ray source and an x-ray detector that are mounted so as to be movable about a center of rotation in a defined arrangement. The medical image data may have projection mappings of the examination object and the medical object. Further, the light guiding facility may be arranged on the x-ray source or the x-ray detector.

In one embodiment, the imaging device may be configured as a medical x-ray device (e.g., medical C-arm x-ray device). The imaging device (e.g., the x-ray device) may have an x-ray source and an x-ray detector that are mounted in a defined arrangement (e.g., on a shared C-arm so as to be movable, such as rotatable about a center of rotation, such as an isocenter). In one embodiment, the image data may have projection mappings of the examination object and the medical object. The x-ray source may emit an x-ray beam bundle in order to record the projection mappings. Further, the x-ray detector may detect the x-ray beam bundle after interaction between the x-ray beam bundle and the examination object and the medical object and provide a corresponding signal (e.g., on the processing unit).

In one embodiment, the light guiding facility may be arranged on the x-ray source or the x-ray detector. For example, the light guiding facility may be fastened on the x-ray source or the x-ray detector. Alternatively or in addition, the light guiding facility may be integrated at least in part (e.g., completely) into the x-ray source or the x-ray detector. The light guiding facility may, for example, be moved (e.g., uniformly or homogenously) with a movement (e.g., a translation and/or a rotation) of the defined arrangement between the x-ray source and x-ray detector. An inherent registration between a coordinate system of the light guiding facility and a coordinate system of the imaging device may be achieved as a result.

In a further embodiment of the system, the light guiding facility may emit the light distribution in the operating state of the apparatus, having a light array. In one embodiment, the light array may run at least virtually through a focal point of the x-ray source and intersect a detection area of the x-ray detector.

The light array may be formed by a number of light beams that run in each case within a layer (e.g., a plane, such as in the manner of an array and/or parallel to one another). A straight line may be projected from the light guiding facility by emitting the predefined light distribution, having the light array.

The x-ray source may have the focal point that may describe a spatial position of a convergence of x-rays of the x-ray beam bundle. Alternatively or in addition, the focal point may describe a spatial position of a diaphragm that is arranged on the x-ray source.

Further, the x-ray detector may have a detection area (e.g., a detection layer and/or detection plane of a flat-panel detector and/or a detection line of a line detector). The detection area may refer to the x-ray beam-sensitive area of the x-ray detector (e.g., a surface of the x-ray detector), within which the x-rays emitted by the x-ray source may be detected by the x-ray detector.

In one embodiment, the light guiding facility may emit the light distribution, having the light array, in the operating state such that the light array runs at least virtually (e.g., with unhindered virtual continuation of the light array) through the focal point of the x-ray source, and at least virtually intersects the detection area of the x-ray detector. For example, the light array (e.g., a layer or plane of the light array) may include the focal point of the x-ray source at least virtually. Further, the light array may intersect the detection area (e.g., including a number of detector pixels) of the x-ray detector (e.g., along a line or a rectangle of the detector pixels).

In one embodiment, an inherent correspondence (e.g., inherent registration) may herewith be established between the illuminable detector pixels and the predefined light distribution (e.g., the light array).

In a further embodiment of the system, the system may also include the medical object that is configured as a trocar and/or puncture needle and/or endoscope and/or implant.

The embodiment may enable an alignment of the medical object configured as a trocar and/or puncture needle and/or endoscope and/or implant that is intuitive and x-ray dose-efficient (e.g., in an orthopedic intervention).

In a further embodiment of the system, the identification of the positioning information may include a receiving of a positioning capture signal of an electromagnetic and/or acoustic and/or optical capture unit for capturing the positioning of the medical object. As an alternative or in addition, the identification of the positioning information may include an identification of a mapping of the medical object in the image data.

The capture unit may have an electromagnetic and/or acoustic and/or optical sensor that is configured to capture the positioning (e.g., a spatial position and/or orientation and/or pose) of the medical object. In one embodiment, the sensor may be configured to capture the (e.g., instantaneous) positioning of the medical object (e.g., a predefined section of the medical object). For example, the sensor may capture the positioning of the medical object with respect to the examination object (e.g., in a coordinate system of the examination object). For this purpose, the sensor may be integrated into the medical object (e.g., a distal section of the medical object or arranged at distance from the medical object). Further, the capture unit may be configured to provide the positioning capture signal as a function of the captured positioning of the medical object (e.g., having the positioning information).

Alternatively or in addition, the processing unit may be configured to identify the mapping of the medical object in the image data. The identification of the mapping of the medical object may include an identification (e.g., segmentation) of image points of the image data that maps the medical object. The identification of the mapping of the medical object in the image data may take place, for example, on a threshold value basis using a comparison of the image values of the image points with a predetermined threshold value and/or based on geometric features of the medical object (e.g., a contour and/or marker structure) that are mapped in the image data. For example, the medical object may have a (e.g., imaging-visible, such as x-ray opaque) marker structure (e.g., on a proximal section of the medical object). Further, the processing unit may be configured to determine the instantaneous positioning of the medical object (e.g., a spatial position and/or alignment and/or pose) based on the identified mapping of the medical object and to provide the positioning information as a function of the determined positioning. The instantaneous positioning (e.g., an instantaneous rotation about a longitudinal axis of the medical object) may be identifiable in the image data using the marker structure.

The present embodiments relate, in a third aspect, to a method for emitting a light distribution by a light guiding facility. In one embodiment, medical image data that maps an examination object is captured. Further, an alignment element, having a number of at least point-shaped markers, is fastened on a medical object or integrated into the medical object in a defined positional relationship. Further, an item of positioning information relating to a positioning of the medical object is identified. Further, a graphical representation of the medical image data and an item of alignment information is displayed by a representation unit as a function of the positioning information. In one embodiment, the alignment information relating to at least one of the markers has a corresponding virtual continuation of the medical object. Further, the light distribution is emitted such that one of the markers is illuminated by the light distribution if the medical object is aligned according to the corresponding virtual continuation.

The advantages of the method of the present embodiments correspond essentially to the advantages of the apparatus of the present embodiments. Features, advantages, or alternative embodiments mentioned in this regard may likewise also be transferred to the other claimed subject matters and vice versa.

In a further embodiment of the method, the light distribution may be emitted such that a straight line that illuminates one of the markers and a reference point of the medical object is projected when the medical object is aligned according to the corresponding virtual continuation.

In a further embodiment of the method, the medical object may be configured to be essentially rigid and elongated. In one embodiment, the reference point may be arranged along a longitudinal extension direction of the medical object.

The present embodiments relate, in a fourth aspect, to a computer program product with a computer program that may be loaded directly into a memory of a processing unit, with program sections in order to execute all acts of a method of the present embodiments for emitting a light distribution, when the program sections are executed by the processing unit. In one embodiment, the computer program product may include software with a source code that still has to be compiled and bound or which only has to be interpreted, or may include an executable software code that is only to be loaded into the processing unit for execution purposes. The method for emitting a light distribution from the processing unit may be executed in a rapid, identically-repeatable and robust manner by the computer program product. The computer program product is configured such that the computer program product may execute the method acts of the present embodiments by the processing unit.

The computer program product is saved on a computer-readable storage medium, for example, or stored on a network or server, from where the computer program product may be loaded into the processor of a processing unit that may be connected directly to the processing unit or configured as part of the processing unit. Further, control information of the computer program product may be saved on an electronically readable data carrier. The control information of the electronically readable data carrier may be configured so that the electronically readable data carrier carries out a method of the present embodiments when the data carrier is used in a processing unit. Examples of electronically readable data carriers are a DVD, a magnetic tape, or a USB stick, on which electronically readable control information (e.g., software) is saved. When this control information is read by the data carrier and saved in a processing unit, all embodiments of the previously described method may be carried out.

A largely software-based realization is advantageous in that previously used processing units may easily be retrofitted by a software update in order to operate in a manner of the present embodiments. Such a computer program product may include, in addition to the computer program, possibly additional component parts such as, for example, documentation and/or additional components, as well as hardware components, such as, for example, hardware keys (e.g., dongles, etc.) for utilizing the software.

### BRIEF DESCRIPTION OF THE DRAWINGS

Example embodiments are shown in the drawings and are described in more detail below. The same reference characters are used for the same features in the different figures, in which:

FIG. 1 shows a schematic representation of an embodiment of an apparatus for aligning a medical object with respect to an examination object;

FIGS. 2 and 4 show schematic representations of the apparatus and a medical object in different alignments with respect to the examination object;

FIGS. 3 and 5 show schematic representations of graphical representations of medical image data and an item of alignment information, corresponding to the different alignments of the medical object;

FIG. 6 shows a schematic representation of an embodiment of a proposed system;

FIG. 7 shows a schematic representation of embodiments of a method for emitting a light distribution.

### DETAILED DESCRIPTION

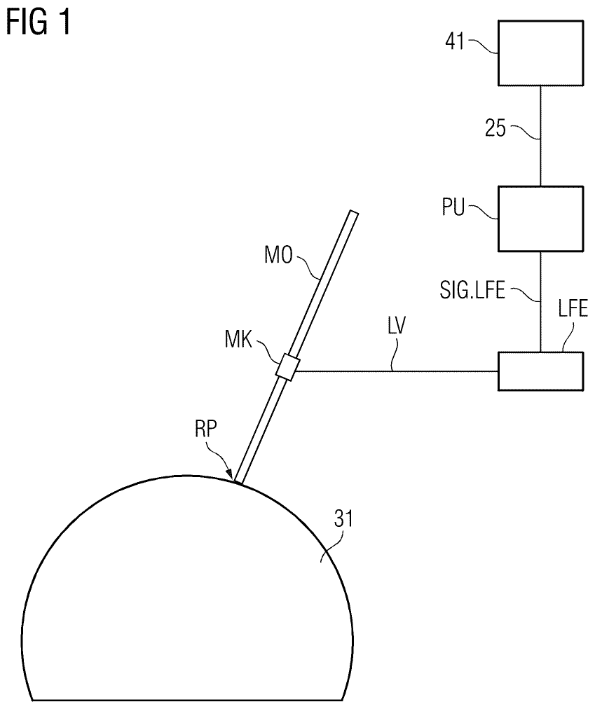

FIG. 1 shows a schematic representation of an embodiment of an apparatus for aligning a medical object MO with respect to an examination object 31. The apparatus may include an alignment element MK, a light guiding facility LFE, a processing unit PU, and a representation unit 41. The alignment element MK may have a number of at least point-shaped markers MK.1, MK.2, MK.3, MK.4, and MK.5. Further, the alignment element MK may be fastened on the medical object MO or integrated into the medical object MO in a defined positional relationship. The processing unit PU may be configured to receive medical image data, having a mapping of the examination object 31. The processing unit PU may further be configured to identify an item of positioning information relating to a positioning of the medical object MO. The identification of the positioning information may include a receiving of a position capture signal of an electromagnetic and/or acoustic and/or optical capture unit for capturing the positioning of the medical object MO. Alternatively or in addition, the identification of the positioning information may include an identification of a mapping of the medical object MO in the image data. Further, the processing unit PU may provide the positioning information on the light guiding facility LFE by a signal S.LFE. The representation unit 41 may be configured to display a graphical representation of the medical image data and an item of alignment information. For this purpose, the processing unit PU may provide a corresponding signal 25 to the representation unit 41. The alignment information may have a virtual continuation of the medical object MO that corresponds to the markers in each case. In one embodiment, the virtual continuations may correspond to virtual arrangements of the medical object MO. The light guiding facility LFE may be configured to emit a predefined light distribution LV (e.g., as a function of the positioning information). Further, the medical object MO may be arranged on or in the examination object 31 in an operating state. Further, the representation unit 41 in the operating state may display the graphical representation of the medical image data and the alignment information. Further, in the operating state of the apparatus, the light guiding facility LFE may emit the light distribution LV (e.g., as a function of the positioning information), such that one of the markers MK.1 to MK.5 is illuminated by the light distribution LV when the medical object MO is aligned according to the corresponding virtual continuation.

In one embodiment, the medical object MO may be configured to be essentially rigid and elongated. The reference point RP may be arranged along a longitudinal extension direction of the medical object MO (e.g., on a distal section of the medical object MO).

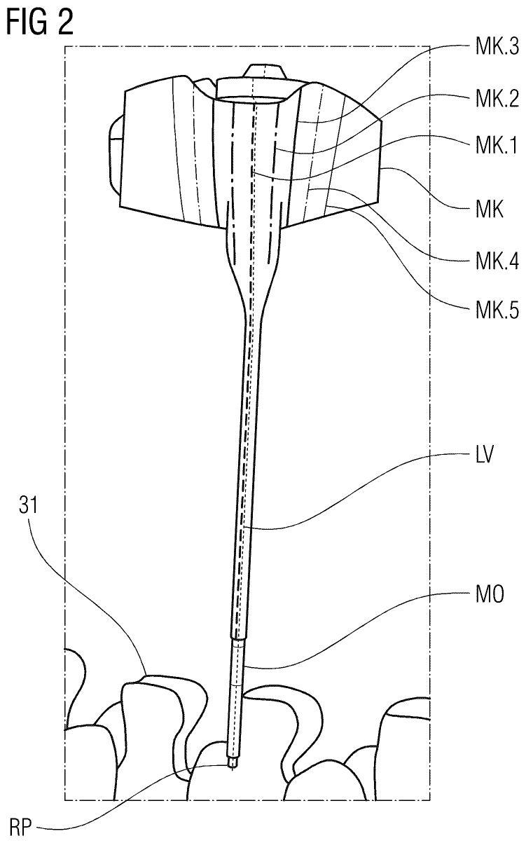

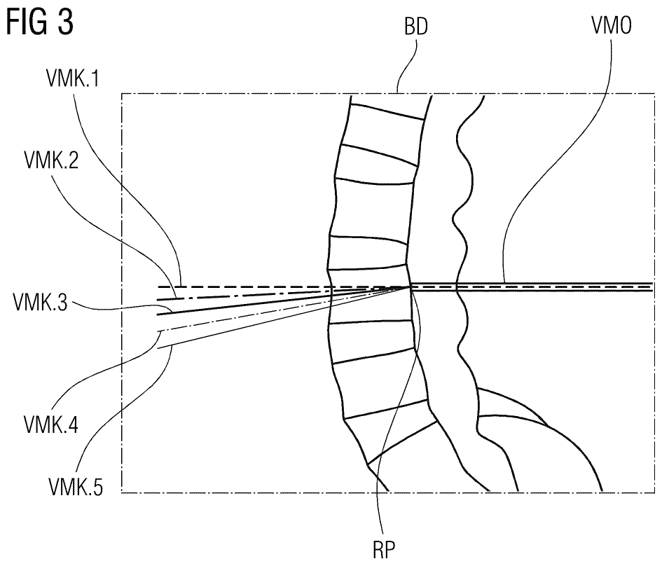

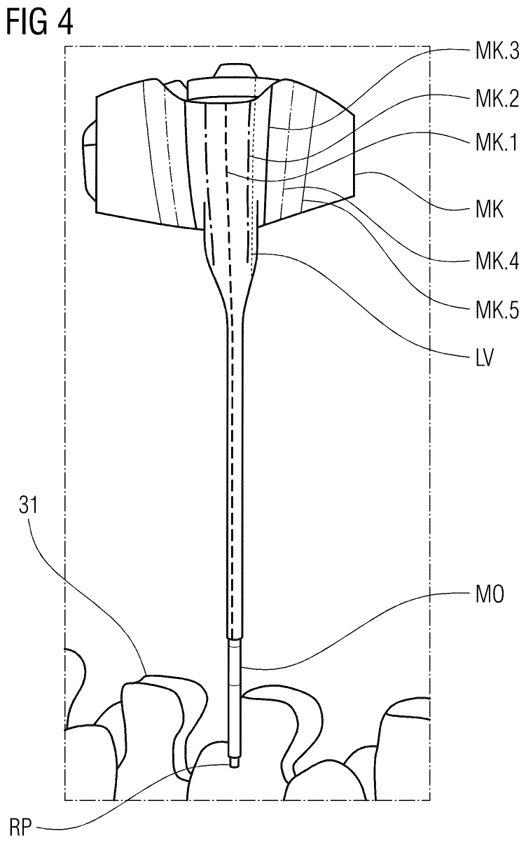

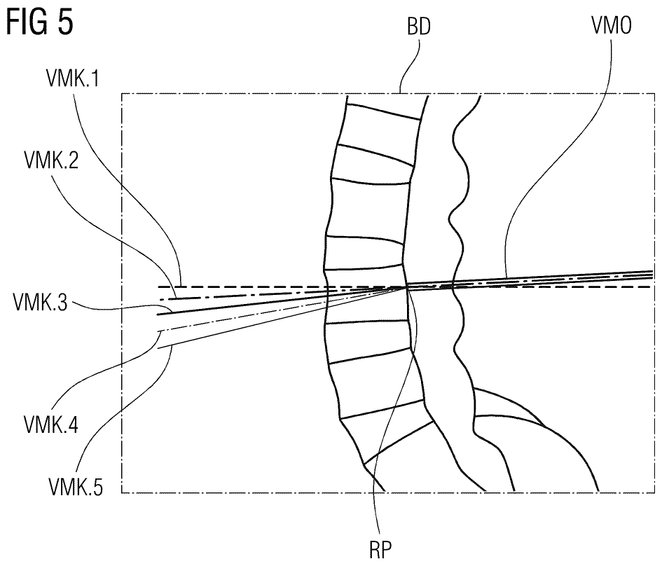

FIGS. 2 and 4 show schematic representations of the apparatus and the medical object MO in various alignments with respect to the examination object 31. FIGS. 3 and 5 show schematic representations of graphical representations of medical image data BD and an item of alignment information that corresponds to the different alignments of the medical object MO shown in FIGS. 2 and 4. For example, the representation in FIG. 2 corresponds to the representation in FIG. 3, and the representation in FIG. 4 corresponds to the representation in FIG. 5.

In one embodiment, the markers MK.1 to MK.5 may have an optically distinguishable property (e.g., a color coding). Further, the number of markers MK.1 to MK.5 may be arranged essentially on a shared flat side of the alignment element MK. In one embodiment, the virtual continuations VMK.1 to VMK.5, which correspond to the markers MK.1 to MK.5 in each case, have an optically distinguishable property. In one embodiment, the optically distinguishable property of the virtual continuations VMK.1 to VMK.5 may correspond to the optically distinguishable property of the markers MK.1 to MK.5 in each case (e.g., by a color code match).

In one embodiment, the number of markers MK.1 to MK.5 may be configured as two-dimensional (2 D) lines within a plane in each case, where the planes intersect along a shared straight line that forms a movement axis of the medical object MO with respect to the examination object and not an object axis of the medical object MO.

As shown in FIGS. 2 and 4, the light guiding facility LFE may emit the light distribution LV in the operating state such that a straight line is projected. The light distribution LV illuminates one of the markers and a reference point RP of the medical object MO when the medical object MO is aligned according to the corresponding virtual continuation. FIG. 2 shows schematically how the medical object MO is aligned according to the virtual continuation VMK.1. In one embodiment, the light distribution LV may illuminate the corresponding marker MK.1. FIG. 4 shows schematically how the medical object MO is aligned according to the virtual continuation VMK.2. In one embodiment, the light distribution LV may illuminate the corresponding marker MK.2.

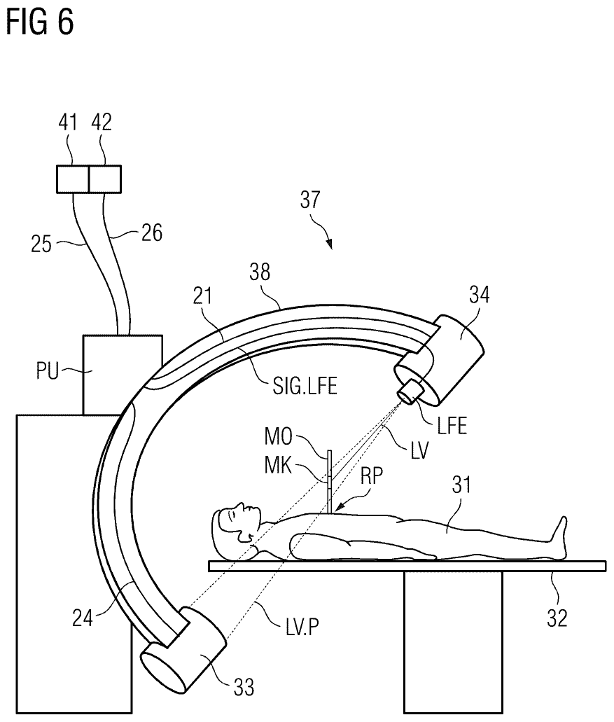

FIG. 6 shows a schematic representation of an embodiment of a system. The system may include one embodiment of an apparatus and a medical imaging device. Further, the system may include the medical object MO that may be configured as a trocar and/or puncture needle and/or endoscope and/or implant. A medical C-arm x-ray device 37 for a medical imaging device is shown by way of example in FIG. 6. The C-arm x-ray device 37 may have an x-ray detector 34 and an x-ray source 33 that are arranged in a defined arrangement on a C-arm 38. The C-arm 38 of the C-arm x-ray device 37 may be mounted so as to be movable about one or more axes. For example, the defined arrangement of x-ray source 33 and x-ray detector 34 may be mounted so as to be movable about a rotation center. Further, the reference point RP may be arranged in the operating state in the center of rotation (e.g., at the same distance between the x-ray source 33 and x-ray detector 34). This may take place, for example, by initial positioning (e.g., arrangement) of a longitudinal axis of the medical object MO in a parallel alignment of the defined arrangement of x-ray source and x-ray detector (bullseye view). The defined arrangement may hereafter be repositioned relative to the medical object in a progression view.

In one embodiment, the light guiding facility LFE may be arranged on the x-ray detector 34. The processing unit PU may send a signal 24 to the x-ray source 33 in order to record the medical image data BD (e.g., at least one projection mapping of the examination object 31 positioned on a patient support apparatus 32 and the medical object MO arranged thereupon). The x-ray source 33 may then emit an x-ray beam bundle. When the x-ray beam bundle impacts a surface of the x-ray detector 34, after interaction with the examination object 31, the x-ray detector 34 may send a signal 21 to the processing unit PU. The processing unit PU may capture the image data BD using the signal 21.

The C-arm x-ray device 37 may further have an input unit 42 (e.g., a keyboard). The input unit 42 may be integrated into the representation unit 41 (e.g., with a capacitive and/or resistive input representation). The input unit 42 may be configured to capture a user input. For this purpose, the input unit 42 may send a signal 26 to the processing unit PU, for example.

In one embodiment, the light guiding facility LFE may emit the light distribution LV in the operating state of the apparatus, having a light array LV.P. In one embodiment, the light array LV.P may run at least virtually through a focal point of the x-ray source 33 and intersect a detection area of the x-ray detector 34.

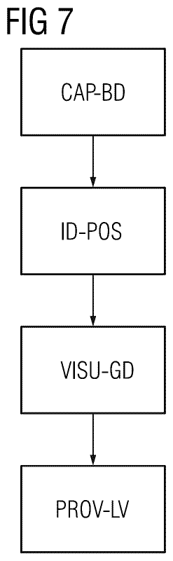

FIG. 7 shows a schematic representation of an embodiment of a method for emitting PROV-LV a light distribution LV. In a first act, the medical image data, which maps the examination object 31, may be captured CAP-BD. In one embodiment, the image data may be captured by a medical imaging device, having an x-ray source and an x-ray detector. In one embodiment, the alignment element MK, having the number of at least point-shaped markers MK.1 to MK.5, may be fastened on the medical object MO or integrated into the medical object MO in a defined positional relationship. In a further act, the positioning information relating to the positioning of the medical object MO may be identified ID-POS. In a further act, the graphical representation of the medical image data and the alignment information may be displayed VISU-GD by the representation unit 41 as a function of positioning information. The alignment information may have a virtual continuation VMK.1 to VMK.5 of the medical object MO that corresponds to at least one of the markers MK.1 to MK.5. In a further act, the light distribution LV (e.g., as a function of the positioning information) may be emitted PROV-LV such that one of the markers MK.1 to MK.5 is illuminated by the light distribution LV when the medical object MO is aligned according to the corresponding virtual continuation VMK.1 to VMK.5.

In one embodiment, the light distribution LV may be emitted PROV-LV such that a straight line is projected. The light distribution LV illuminates one of the markers and the reference point RP of the medical object MO when the medical object MO is aligned according to the corresponding virtual continuation.

The medical object MO may be configured to be essentially rigid and elongated. In one embodiment, the reference point RP may be arranged along the longitudinal extension direction of the medical object MO.

The schematic representations contained in the figures described do not map in any way to scale or ratio.

The method described in detail above and the apparatuses shown are only example embodiments that may be modified by the person skilled in the art in a variety of ways without departing from the scope of the invention. Further, the use of the indefinite article “a” or “an” does not rule out that the relevant features may also be present a number of times. Similarly, the terms “unit” and “element” do not rule out that the relevant components consist of a number of interacting subcomponents that may possibly also be distributed spatially.

In the context of the present application, the expression “based on” may be understood, for example, in the context of the expression “using”. For example, a wording that is generated (alternatively: ascertained, determined, etc.) as a result of a first feature based on a second feature does not rule out that the first feature may be generated (alternatively: ascertained, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for aligning a medical object with respect to an examination object, the apparatus comprising:
an alignment element;
a light guiding facility;
a processing unit; and
a representation unit,
wherein the alignment element has a number of at least point-shaped markers and is fastenable on the medical object or integrated into the medical object in a defined positional relationship,
wherein the processing unit is configured to:
receive medical image data having a mapping of the examination object; and
identify an item of positioning information relating to a positioning of the medical object,
wherein the representation unit is configured to show a graphical representation of the medical image data and an item of alignment information as a function of the positioning information,
wherein the item of alignment information relating to the number of at least point-shaped markers has in each case a corresponding virtual continuation of the medical object,
wherein the virtual continuations correspond to virtual arrangements of the medical object,
wherein the light guiding facility is configured to emit a predefined light distribution, and
wherein in an operating state of the apparatus:
the medical object is arranged on or in the examination object;
the representation unit is configured to show the graphical representation of the medical image data and the alignment information; and
the light guiding facility is configured to emit the light distribution so that one marker of the number of at least point-shaped markers is illuminated by the light distribution when the medical object is aligned according to the corresponding virtual continuation.

2. The apparatus of claim 1, wherein the number of at least point-shaped markers have an optically distinguishable property,
wherein the virtual continuations in the graphical representation of the alignment information have an optically distinguishable property, and
wherein the optically distinguishable property of the virtual continuations corresponds in pairs to the optically distinguishable property of one of the number of at least point-shaped markers in each case.

3. The apparatus of claim 1, wherein the number of at least point-shaped markers are configured as two-dimensional (2 D) lines within a plane in each case, and
wherein the planes intersect along a shared straight line that forms a movement axis of the medical object with respect to the examination object and no object axis of the medical object.

4. The apparatus of claim 1, wherein the number of at least point-shaped markers are arranged essentially on a shared side of the alignment element.

5. The apparatus of claim 1, wherein the light guiding facility is configured to emit the light distribution in the operating state such that a straight line is projected that illuminates one of the markers and a reference point of the medical object, when the medical object is aligned according to the corresponding virtual continuation.

6. The apparatus of claim 5, wherein the medical object is configured to be essentially rigid and elongated, and
wherein the reference point is arranged along a longitudinal extension direction of the medical object.

7. The apparatus of claim 1, wherein the light-guiding facility is configured to emit the predefined light distribution in the operating state as a function of the positioning information.

8. A system comprising:
an apparatus for aligning a medical object with respect to an examination object, the apparatus comprising:
an alignment element;
a light guiding facility;
a processing unit; and
a representation unit, wherein the alignment element has a number of at least point-shaped markers and is fastenable on the medical object or integrated into the medical object in a defined positional relationship, wherein the processing unit is configured to receive medical image data having a mapping of the examination object, and identify an item of positioning information relating to a positioning of the medical object, wherein the representation unit is configured to show a graphical representation of the medical image data and an item of alignment information as a function of the positioning information, wherein the item of alignment information relating to the number of at least point-shaped markers has in each case a corresponding virtual continuation of the medical object, wherein the virtual continuations correspond to virtual arrangements of the medical object, wherein the light guiding facility is configured to emit a predefined light distribution, and wherein in an operating state of the apparatus, the medical object is arranged on or in the examination object, the representation unit is configured to show the graphical representation of the medical image data and the alignment information, and the light guiding facility is configured to emit the light distribution so that one marker of the number of at least point-shaped markers is illuminated by the light distribution when the medical object is aligned according to the corresponding virtual continuation; and
a medical imaging device,
wherein the medical imaging device is configured to record the medical image data.

9. The system of claim 8, wherein the medical imaging device has an x-ray source and an x-ray detector that is mounted so as to be movable about a center of rotation in a defined arrangement,
wherein the medical image data has projection mappings of the examination object and the medical object, and
wherein the light guiding facility is arranged on the x-ray source or the x-ray detector.

10. The system of claim 9, wherein the light guiding facility is configured to emit the light distribution in the operating state of the apparatus, having a light array, and
wherein the light array runs at least virtually through a focal point of the x-ray source and intersects a detection area of the x-ray detector.

11. The system of claim 8, further comprising the medical object, which is configured as a trocar, a puncture needle, an endoscope, an implant or any combination thereof.

12. The system of claim 8, wherein:
the identification of the positioning information comprises receipt of a positioning capture signal of an electromagnetic and/or acoustic and/or optical capture unit for capturing the positioning of the medical object;
the identification of the positioning information comprises identification of a mapping of the medical object in the image data; or
a combination thereof.

13. A method for emitting a light distribution, the method comprising:
capturing medical image data that maps an examination object, wherein an alignment element, having a number of at least point-shaped markers, is fastened on a medical object or integrated into the medical object in a defined positional relationship;
identifying an item of positioning information relating to a positioning of the medical object;
showing a graphical representation of the medical image data and alignment information as a function of the positioning information using a representation unit, wherein the alignment information relating to at least one of the number of at least point-shaped markers has a corresponding virtual continuation of the medical object; and
emitting the light distribution such that one of the number of at least point-shaped markers is illuminated by the light distribution when the medical object is aligned in accordance with the corresponding virtual continuation.

14. The method of claim 13, wherein the light distribution is emitted such that a straight line is projected, the light distribution illuminating one of the number of at least point-shaped markers and a reference point of the medical object when the medical object is aligned in accordance with the corresponding virtual continuation.

15. The method of claim 14, wherein the medical object is configured to be essentially rigid and elongated, and
wherein the reference point is arranged along a longitudinal extension direction of the medical object.
